SEQUENCE LISTING (35 sequences)

SEQ ID NO: 1 - DNA, 538 bp, Artificial Sequence
  Description of Artificial Sequencesynthetic
catggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggact

SEQ ID NO: 2 - DNA, 637 bp, Artificial Sequence
  Description of Artificial Sequencesynthetic
attcagctttcgttcgtatcatcggtttcgacaacgttcgtcaagttcaatgcatcagtttcattgcgcacacaccagaatcctactgagttcgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgccaattgattgacaac

SEQ ID NO: 3 - DNA, 795 bp, Artificial Sequence
  Description of Artificial Sequencesynthetic
atgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgactcatggcgaagcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctga

SEQ ID NO: 4 - DNA, 403 bp, Artificial Sequence
  Description of Artificial Sequencesynthetic
caattatacatttaatacgcgatagaaaacaaaatatagcgcgcaaactaggataaattatcgcgcgcggtgtcatctatgttactagatcggggatcgggccactcgaccaagctcctcatctaagcccccatttggacgtgaatgtagacacgtcgaaataaagatttccgaattagaataatttgtttattgctttcgcctataaatacgacggatcgtaatttgtcgttttatcaaaatgtactttcattttataataacgctgcggacatctacatttttgaattgaaaaaaaattggtaattactctttctttttctccatattgaccatcatactcattgctgatccatgtagatttcccggacatgaagccatttacaattgaatatatcctgcc

SEQ ID NO: 5 - DNA, 96 bp, Artificial Sequence
  Description of Artificial Sequencesynthetic
gaggacacagaaaaatttgctacattgtttcacaaacttcaaatattattcatttatttgtcagctttcaaactctttgtttcttgtttgttgatt

SEQ ID NO: 6 - DNA, 265 bp, Artificial Sequence
  Description of Artificial Sequencesynthetic
gatcatgagcggagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattaga

SEQ ID NO: 7 - DNA, 22 bp, Artificial Sequence
  Description of Artificial Sequencesynthetic
caacgttcgtcaagttcaatgc

SEQ ID NO: 8 - DNA, 26 bp, Artificial Sequence
  Description of Artificial Sequencesynthetic
tgccataatactcgaactcagtagga

SEQ ID NO: 9 - DNA, 26 bp, Artificial Sequence
  Description of Artificial Sequencesynthetic
tcagtttcattgcgcacacaccagaa

SEQ ID NO: 10 - DNA, 20 bp, Artificial Sequence
  Description of Artificial Sequencesynthetic
cccgatcgttcaaacatttg

SEQ ID NO: 11 - DNA, 32 bp, Artificial Sequence
  Description of Artificial Sequencesynthetic
cgtaattcaacagaaattatatgataatcatc

SEQ ID NO: 12 - DNA, 30 bp, Artificial Sequence
  Description of Artificial Sequencesynthetic
tcttaagattgaatcctgttgccggtcttg

SEQ ID NO: 13 - DNA, 17 bp, Artificial Sequence
  Description of Artificial Sequencesynthetic
ggatcgggccactcgac

SEQ ID NO: 14 - DNA, 26 bp, Artificial Sequence
  Description of Artificial Sequencesynthetic
tctttatttcgacgtgtctacattca

SEQ ID NO: 15 - DNA, 27 bp, Artificial Sequence
  Description of Artificial Sequencesynthetic
aagctcctcatctaagcccccatttgg

SEQ ID NO: 16 - DNA, 17 bp, Artificial Sequence
  Description of Artificial Sequencesynthetic
cgatgacgcgggacaag

SEQ ID NO: 17 - DNA, 20 bp, Artificial Sequence
  Description of Artificial Sequencesynthetic
ggctgagtggctccttcaac

SEQ ID NO: 18 - DNA, 29 bp, Artificial Sequence
  Description of Artificial Sequencesynthetic
tttacgtttggaactgacagaaccgcaac

SEQ ID NO: 19 - DNA, 20 bp, Artificial Sequence
  Description of Artificial Sequencesynthetic
agatggattgcacgcaggtt

SEQ ID NO: 20 - DNA, 22 bp, Artificial Sequence
  Description of Artificial Sequencesynthetic
gtgcccagtcatagccgaatag

SEQ ID NO: 21 - DNA, 20 bp, Artificial Sequence
  Description of Artificial Sequencesynthetic
ctctccacccaagcggccgg

SEQ ID NO: 22 - DNA, 20 bp, Artificial Sequence
  Description of Artificial Sequencesynthetic
ccggccgcttgggtggagag

SEQ ID NO: 23 - DNA, 17 bp, Artificial Sequence
  Description of Artificial Sequencesynthetic
agagcttggcggcgaat

SEQ ID NO: 24 - DNA, 16 bp, Artificial Sequence
  Description of Artificial Sequencesynthetic
aatcgggagcggcgat

SEQ ID NO: 25 - DNA, 24 bp, Artificial Sequence
  Description of Artificial Sequencesynthetic
tgaccgcttcctcgtgctttacgg

SEQ ID NO: 26 - DNA, 32 bp, Artificial Sequence
  Description of Artificial Sequencesynthetic
acttcaaatattattcatttatttgtcagctt

SEQ ID NO: 27 - DNA, 25 bp, Artificial Sequence
  Description of Artificial Sequencesynthetic
ttgtttcttgtttgttgattagatc

SEQ ID NO: 28 - DNA, 27 bp, Artificial Sequence
  Description of Artificial Sequencesynthetic
caatgccataatactcgaactcagtag

SEQ ID NO: 29 - DNA, 660 bp, Artificial Sequence
  Description of Artificial Sequencesynthetic
atggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccgaaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaa

SEQ ID NO: 30 - DNA, 861 bp, Artificial Sequence
  Description of Artificial Sequencesynthetic
atgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaa

SEQ ID NO: 31 - DNA, 401 bp, Artificial Sequence
  Description of Artificial Sequencesynthetic
attcttttctttttttttcttttctctctcccccgttgttgtctcaccatatccgcaatgacaaaaaaaatgatggaagacactaaaggaaaaaattaacgacaaagacagcaccaacagatgtcgttgttccagagctgatgaggggtatcttcgaacacacgaaactttttccttccttcattcacgcacactactctctaatgagcaacggtatacggccttccttccagttacttgaatttgaaataaaaaaagtttgccgctttgctatcaagtataaatagacctgcaattattaatcttttgtttcctcgtcattgttctcgttccctttcttccttgtttctttttctgcacaatatttcaagctataccaagcatacaatcaactccccggg

SEQ ID NO: 32 - DNA, 68 bp, Triticum aestivum
tctagagggccaccaccacggtgcgcgccaagacaaggcaggggagagaaattcgtcaatccgcagca

SEQ ID NO: 33 - DNA, 231 bp, triticum aestivum
aattcgctcctggccatggagctgcttctgtctctgggttcacaagtctcggtgtctccggtatcctccaatggagtctggtctgtgtctgtcgttgcctgactgtctttgtttctgtaccatactgtgatgcagtgttatcgtttgtatcttcaaacttctgctggtgtggagcagctttggtgaactatgaataagtgagcggagatctgttgtgtgttttttggatcc

SEQ ID NO: 34 - DNA, 284 bp, Artificial Sequence
  Description of Artificial Sequencesynthetic
taacatctacaaattgccttttcttatcgaccatgtacgtaagcgcttacgtttttggtggacccttgaggaaactggtagctgttgtgggcctgtggtctcaagatggatcattaatttccaccttcacctacgatggggggcatcgcaccggtgagtaatattgtacggctaagagcgaatttggcctgtagacctcaattgcgagctttctaatttcaaactattcgggcctaacttttggtgtgatgatgctgactggcaggatatataccgttgtaatt

SEQ ID NO: 35 - DNA, 253 bp, Agrobacterium tumafaciens
gatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

(19) United States
(12) Patent Application Publication
(10) Pub. No.: US 2006/0127889 A1
(43) Pub. Date: Jun. 15, 2006

(54) Method for assessing transgene expression and copy number

(76) Inventors: Stanton B. Dotson, Chesterfield, MO (US); Jon J. Schmuke, St. Louis, MO (US); John C. Anderson, University City, MO (US)

Correspondence Address: Lawrence M. Lavin, Jr.;Patent Department, E2NA, Monsanto Company, 800 N. Lindbergh Boulevard, St. Louis, MO, 63167, US

(21) Appl. No.: 09/912,968

(22) Filed: Jul. 25, 2001

Related U.S. Application Data
(60) Provisional application No. 60/220,571, filed on Jul. 25, 2000.

(51) Int. Cl. C12Q 1/68

(52) U.S. Cl. 435/6

(57) ABSTRACT

The invention relates to the field of genetic engineering. More particularly the invention relates to a method of quantitating transgenes or transgene expression by using sequences commonly included in transformation plasmids or vectors. The invention also provides primers and probes which can be used with this method.

### FIELD OF THE INVENTION

Disclosed herein are inventions relating to genetic engineering, more particularly to methods of quantitating transgenes or transgene expression by using sequences commonly included in transformation plasmids or vectors. Also disclosed are oligonucleotide primers and probes which can be used with these methods.

### INCORPORATION OF SEQUENCE LISTING

This application contains a sequence listing, which is contained on three identical CD-ROMs: two copies of a sequence listing (Copy 1 and Copy 2) and a sequence listing Computer Readable Form (CRF), all of which are herein incorporated by reference. All three CD-ROMs each contain one file called “Method.APP.doc” which is 54,272 bytes in size and was created on Jul. 13, 2001.

### BACKGROUND

Progress in molecular biology has enabled the seemingly routine insertion of foreign genes into plants, animals and microorganisms, usually with the intention of conferring desirable traits in the receiving (host) organism. For example, a gene of interest which encodes a protein relating to a specific trait in one species may be introduced into another species. In a successful transformation, enzymes in the host organism use the foreign gene which is made up of a DNA sequence as a template to synthesize a single stranded messenger nucleic acid molecule (mRNA) chain which serves as a code that is read by other cellular factors to produce a new protein in a process called translation. The new protein may cause the host organism to exhibit a new trait.

Alternatively, a foreign gene may be inserted into a bacterium, plant or other organism for the purpose of manufacturing large amounts of protein such as interferon, growth hormone, or insulin. In this case, the intent is not to change any traits in the host but to use the host as a factory.

In most cases, vectors are used to introduce a foreign gene of interest into a host organism. A vector can comprise DNA sequences originating from a virus, plasmid, cosmid, plasmid or bacteriophage into which the foreign gene of interest can be integrated. Vectors can also be synthesized by chemical or enzymatic means. Vectors may contain native sequences which enable them to self replicate or may integrate in a host genome and replicate with a host genome A gene of interest in a vector can be operably linked to a promoter and other regulatory sequences to enhance or enable mRNA to be formed from the foreign gene or which stabilize the mRNA molecule.

The leading end of a gene sequence where translation starts is by convention called the 5′ end; the other end of the gene is called the 3′ end. Different regulatory sequences may be added to different parts of a foreign gene. Commonly, a promoter sequence is operably linked upstream (i.e. at the 5′ end) of the gene of interest to enable and enhance transcription (i.e. the formation of RNA from the DNA template). Promoters are often selected from a group of well-developed promoters of predictable reliablility and performance. Other sequences, such as “5′untranslated leader sequences” may also be operably linked to the gene of interest and are often included as part of the promoter element. These can act to improve the efficiency of protein translation from the template mRNA and may increase or maintain mRNA stability. “3′ untranslated sequences” have also been shown to increase mRNA stability and can act to stop the formation of a mRNA chain from a foreign gene of interest. Sequences referred to as “intron” sequences may be added internally to 5′ untranslated leader sequences to enhance mRNA translation. Such other sequences are all transcribed into RNA along with the foreign gene.

Vectors can also contain one or more “marker” sequences which are used to determine if transformation was successful. Some markers confer antibiotic resistance to aid in determining whether or not transformation occurred. For example, many types of cells die when grown on a medium containing kanamycin. If a number of cells are putatively transformed with a vector containing a marker gene which confers resistance to kanamycin, one can surmise that cells which survive when grown on a kanamycin-containing medium had been successfully transformed by the vector and therefore also contain the foreign gene of interest.

A vector containing a gene of interest can be delivered into a host organism by a variety of methods. For example, a vector can be injected into a cell of a host organism with a thin hollow needle, by electroporation, by gene gun or by an Agrobacterium plasmid. In the case of plants, the gene gun and tumor-inducing Agrobacterium tumefaciens plasmids are commonly used as the delivery mechanism

Vectors can be engineered to stably integrate the foreign gene into a host chromosome or they may be engineered so that the entire vector can reside outside of the host chromosome where it may replicate. Vector selection depends on the purpose for transformation. For example, when the goal of transformation is to manufacture a small amount of protein or mRNA, vectors which transform outside of the chromosomes are often used. Such vectors typically contain all the necessary regulatory sequences for expressing the foreign protein in the cells from which it can be harvested. When a foreign gene is stably introduced into a host genome, the vector may be designed to integrate additional sequences, such as a promoter sequence into the genome along with the foreign gene. When Agrobacterium transformation is used, part of the tumor inducing plasmid, (tDNA) may also be stably integrated into the host genome.

It is often desirable to know if the gene of interest has been successfully transferred into a host or how many copies of a foreign gene were integrated into either the host genome or reside outside of a host genome. Additionally, it may be desirable to test a sample of cells for the presence of any foreign genes. Often, it is important to know if mRNA is actually transcribed and how much mRNA is present in the host.

Conventional techniques to detect and quantitate specific DNA or mRNA molecules use one or more short nucleic acid sequences (oligonucleotides) which can hybridize to the DNA or mRNA. Designing and synthesizing these oligonucleotides for detecting genes of interest and optimizing the conditions for their effective use is time consuming and often the rate limiting step in using quantitative methods.

An object of this invention is to provide methods to provide a rapid, high performance assay for the indirect detection and quantitation of transgenic genes and transgenic expression.

Another object of this invention is to provide kits of oligonucleotides for a rapid, high performance assay for the indirect detection and quantitation of transgenic genes.

### SUMMARY OF THE INVENTION

This invention provides methods for the indirect detection of a transgenic gene of interest which may be present in a host by providing oligonucleotides complementary to vector sequences other than the gene of interest. Using oligonucleotides which hybridize to common vector sequences greatly reduces the time, effort and cost needed to detect a variety of distinct transgenic genes.

There is often a one-to-one correspondance between mRNA which is transcribed from regulatory sequences included in a vector and the mRNA which is transcribed from a transgenic gene of interest. Therefore, another aspect of the invention provides oligonucleotides which are complementary to mRNA transcribed from these vector sequences as a surrogate indicator of the transgenic gene. The oligonucleotides of this invention can be used with conventional quantitative methods to determine the amount of transgenic mRNA that is in the cell.

A more particular aspect of this invention provides a method to detect the presence or absence of a first transgenic nucleic acid molecule in a sample by assay for a second, more common, transgenic nucleic acid molecule. The method comprises hybridizing the second transgenic nucleic acid molecule with at least one oligonucleotide designed to hybridize to the second transgenic nucleic acid molecule. Hybridizing indicates the presence of a first transgenic nucleic acid molecule in the sample.

An additional aspect of this invention provides an amplification kit for the detection of foreign genes comprising at least one primer pair of oligonucleotides and a corresponding probe oligonucleotide which hybridize to the second nucleic acid molecules. In a more preferred aspect of the kit, the oligonucleotides comprise at least 15 bases of sequence which is substantially complementary to a consecutive sequence of a larger sequence e.g. of a common transgenic element including certain promoters, 3′ untranslated regions, tDNA border region, 5′ leader sequences, marker genes, etc. Such common transgenic elements (defined below as “a second nucleic acid molecule”) include those having a sequence selected from the group consisting of SEQ ID NO: 1 to SEQ ID NO: 6 and SEQ ID NO: 29 to SEQ ID NO: 35. In an even more preferred aspect of this invention, the kit comprises oligonucleotide primers and labeled probes selected from the group consisting of SEQ ID NO: 7 to SEQ ID NO: 28 and complementary sequences thereof.

### DETAILED DESCRIPTION OF THE EMBODIMENTS

Transgenic Nucleic Acid Molecules

Nucleic acid sequences of the present invention include plant, animal including mammalian such as human, bovine and porcine, fish, avian, insect, fungal, algal, viral and bacterial nucleic acid molecules.

As used herein a “transgenic nucleic acid molecule” means with reference to a host organism a nucleic acid molecule which has been introduced into the host organism including genes, promoters, regulatory elements, vector elements and fragments thereof. Transgenic nucleic acids molecules may be foreign to the host in that they are not found in the genome of the individual host cell or may be found in a different locus of the host, e.g. with different regulatory elements. Transgenic nucleic acid molecules may be from the same species as the host or from different species.

As used herein a “first transgenic nucleic acid molecule” means a transgenic nucleic acid molecule which is of interest for detection and/or quantitation of copy number or expression. The methods of this invention are particularly useful for detecting first transgenic nucleic acid molecules which are not commonly used components of vectors including commonly used promoters, regulatory elements and markers. While a first transgenic nucleic acid molecule can comprise any DNA sequence which may have been recombined into the genome of a host organism or be contained on a self replicating vector, a first transgenic nucleic acid molecule will preferably comprise an exogenous gene of interest. The first transgenic nucleic acid molecule may be both transcribed and translated, only transcribed or neither transcribed or translated. The first transgenic nucleic acid molecule may be stably integrated into the chromosome of the host along with other DNA sequences comprising second transgenic nucleic acid molecules or may reside as an episome within the cell.

As used herein “second transgenic nucleic acid molecule” means any transgenic nucleic acid molecule which is conveniently used as a surrogate indicator for a “first” transgenic nucleic acid molecule. Thus, a second transgenic nucleic acid molecule may advantageously include any of the more commonly used DNA elements used in recombinant DNA methods, including promoters and regulatory elements for genes of interest, markers, and elements which may be included within a vector or expression cassette containing a first transgenic nucleic acid molecule. A second transgenic nucleic acid molecule may be operably linked to a first transgenic nucleic acid molecule. A second transgenic nucleic acid molecule may be from the same species as the host but is preferably from a different species as the host. A second transgenic nucleic acid molecule can include nucleic acid molecules or fragments of nucleic acid molecules which (a) enable or enhance expression of a first transgenic nucleic acid molecule, (b) enable secretion of the protein that may be translated from the first transgenic nucleic acid molecule, (c) enable incorporation of the first transgenic nucleic acid molecule into a host genome, (d) are used to deliver the first transgenic nucleic acid molecule to a host cell or (e) are used to replicate the first transgenic nucleic acid molecule in the host cell. Second transgenic nucleic acid molecules may additionally include any other sequences desirable to include in a vector or a nucleic acid expression cassette. Any second transgenic nucleic acid molecule can be transcribed and translated, transcribed only or neither transcribed or translated. Second transgenic nucleic acid molecules may include regulatory elements including, but not limited to 5′ untranslated sequences, introns, 3′ untranslated sequences, promoters and enhancers; DNA sequences used for stable integration such as the right and left tDNA border sequences; sequences coding for selectable or screenable markers, signal sequences and vector backbone sequences.

As used herein “sample” means any composition being tested for the presence, expression, copy number or zygosity of a foreign gene of interest. Embodiments of samples include bacteria, cells, tissue, a biological fluid (i.e. blood or serum) or any solution that may contain the foreign gene. The sample may also contain other nucleic acids, as well as any other components, including, but not limited to, proteins, peptides, carbohydrates and any other components, so long a the components of the sample do not interrupt the ability of an oligonucleotide to hybridize with the second transgenic nucleic acid molecule. In certain embodiments of the invention, certain characteristics of the sample composition (i.e. pH, temperature, ionic strength) must be adjusted in order to allow conditions for hybrid formation to occur. The manipulation of such conditions is well known to those skilled in the art.

As used herein “DNA” means both genomic DNA sequence and the corresponding cDNA.

As used herein, “regulatory elements” means nucleic acid sequences that can enhance or stabilize. mRNA transcription or translation. These sequences include, but are not limited to, promoter sequences, enhancer sequences, 5′ untranslated leader sequences (5′ UTR's), 3′ untranslated sequences (3′ UTR's), introns , transcription and translation termination signals and ribosomal binding domains.

As used herein “vector” means a vehicle used for transferring a foreign gene into cells of a host organism. The components of a vector can include a first transgenic nucleic acid molecule and second transgenic nucleic acid molecules.

As used herin “polylinker” means DNA which contains the recognition site(s) for a specific restriction endonuclease. Polylinker may be ligated to the ends of DNA fragments prepared by cleavage with some other enzyme. In particular, a polylinker provides a recognition site for inserting a nucleic acid expression cassette which contains a specific nucleic acid sequence to be expressed. This recognition site may be but is not limited to an endonuuclease site in the polylinker, such as Cla-I, Not-I, Xmal, Bgl-II, Pac-I, Xhol, Nhe I, Sfi-I. A polylinker can be designed so that the unique restriction endonuclease site contains a start codon (e.g. AUG) or stop codon (e.g. TAA, TGA, TCA) and these critical codons are reconstituted when a sequence encoding a protein is ligated into the linker.

As used herein, a “vector backbone sequence” means a piece of DNA containing at least a region of DNA that enables a vector to replicate (origin of replication) and a selectable marker gene (e.g., an antibiotic resistance gene), optionally, site specific recombination elements, and, optionally, a polylinker region.

As used herein “site specific recombination element” means a piece of DNA arranged in such a manner that a recombinase protein acts to intramolecularly or intermolecularly recombine DNA within the site specific recombination element. (E.g. Saccharomyces cerevisiae Cre recombines DNA at 34 bp sites called loxp. Each loxP consists of two 13 bp inverted repeats (recombinase-binding sites) flanking an 8 bp core region. Intramolecular recombination results in either excision of intervening DNA if the sites are directly repeated, or DNA inversion if the sites are in opposing orientations. Intermolecular recombination results in integration of a circular DNA into another DNA molecule, or reciprocal translocation if both DNAs are linear).

As used herein “episome” means a a low molecular weight DNA molecule that resides in a cell separated from the cell's chromosome(s). Episomes can replicate independently of the host cell chromosomes, and can be transmitted to daughter cells.

As used herein “stable transformation” means the introduction and integration of a transgenic nucleic acid molecule into the genome of a transformed cell.

As used herein “nucleic acid expression cassette” means a group of nucleic acid molecules, e.g. a first transgenic nucleic acid molecule and at least one second transgenic nucleic acid molecule. The nucleic acid expression cassette is positionally and sequentially oriented within a vector such that the nucleic acid molecules in the cassette can be transcribed into mRNA, and when necessary, translated into a protein in the transformed tissue or cell. Preferably, the nucleic acid expression cassette has 3′ and 5′ ends adapted for ready insertion into a vector polylinker, e.g., it has restriction endonuclease sites at each end. Nucleic acid expression cassettes may be inserted into vectors appropriate for stable integration or episomal existence in the host organism.

The terms “in operable combination”, “in operable order” and “operably linked” as used herein refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

The term “oligonucleotides” as used herein means short nucleic acid molecules useful, e.g. for hybridizing probes, or amplification primers. Oligonucletide molecules comprise two or more nucleotides, i.e. deoxyribonucleotides or ribonucleotides, preferably more than five and up to 30 or more. The exact size will depend on many factors, which in turn depend on the ultimate function or use of the oligonucleotide. Oligonucleotides can comprise ligated natural nucleic molecules acids or synthesized nucleic acid molecules and comprise between 5 to 150 nucleotides or between about 15 and about 100 nucleotides, or preferably up to 100 nucleotides, and even more preferably between 15 to 30 nucleotides or most preferably between 18-25 nucleotides, identical or complementary to a second transgenic nucleic acid molecule.

This invention provides oligonucleotides specific for second transgenic nucleic acid molecules. Such primers for use in polymerase chain reaction (PCR) are preferably designed with the goal of amplifying nucleic acids from either the 3′ or the 5′ end of a second transgenic nucleic acid molecule or a fragment of a second transgenic nucleic acid molecule.

The term “primer” as used herein means an oligonucleotide which is capable of acting as a point of initiation of synthesis when placed under conditions in which polynucleotide synthesis of a primer extension product which is complementary to a nucleic acid strand is induced, i.e., in the presence of nucleotides and an agent for polymerization such as DNA polymerase and at a suitable temperature and pH. A primer can be derived from a naturally occurring molecule, e.g. by restriction digest, or produced synthetically. The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the agent for polymerization. The exact lengths of the primers will depend on many factors, including temperature and source of primer. For example, depending on the complexity of the target sequence, the oligonucleotide primer typically contains at least 15, more preferably 18 nucleotides, which are at least substantially identical or complementary to the template. Short primer molecules generally require cooler temperatures to form sufficiently stable hybrid complexes with the template.

The primers herein are selected to be “substantially” complementary to the different strands of each specific sequence to be amplified. This means that the primers must be sufficiently complementary to hybridize with their respective strands. Therefore, the primer sequence need not reflect the exact sequence of the template. For example, a non-complementary nucleotide fragment may be attached to the 5′ end of the primer, with the remainder of the primer sequence being complementary to the strand. Alternatively, non-complementary bases or longer sequences can be interspersed into the primer, provided that the primer sequence has sufficient complementarity with the sequence of the strand to be amplified to hybridize therewith and thereby form a template for synthesis of the extension product of the other primer. Computer generated searches using programs such as Primer3 (www-genome.wi.mit.edu/cgi-bin/primer/primer3.cgi), STSPipeline (www-genome.wi.mit.edu/cgi-bin/www-STS Pipeline), or GeneUp (Pesole et al., BioTechniques 25:112-123 (1998)), for example, can be used to identify potential PCR primers. Exemplary primers include primers that are 18 to 50 bases long, where at least between 18 to 25 bases are identical or complementary to at least 18 to 25 bases segment of the template sequence.

This invention also contemplates and provides primer pairs for amplification of nucleic acid molecules representing second transgenic nucleic acid molecules. As used herein “primer pair” means a set of two oligonucleotide primers based on two separated sequence segments of a target nucleic acid sequence. One primer of the pair is a “forward primer” or “5′ primer” having a sequence which is identical to the more 5′ of the separated sequence segments. The other primer of the pair is a “reverse primer” or “3′ primer” having a sequence which is complementary to the more 3′ of the separated sequence segments. A primer pair allows for amplification of the nucleic acid sequence between and including the separated sequence segments. Optionally, each primer pair can comprise additional sequences, e.g. universal primer sequences or restriction endonuclease sites, at the 5′ end of each primer, e.g. to facilitate reamplification of the target nucleic acid sequence. Useful universal primer sequence can comprise sequences from common vector elements.

The term “probe” as used herein means a labeled oligonucleotide which forms a duplex structure with a sequence in another nucleic acid, due to complementarity of at least one sequence in the probe with a sequence in the other nucleic acid.

The term “corresponding probe” as used herein means that the probe anneals between the forward and reverse primers to which it corresponds.

The term “label” as used herein refers to any atom or molecule or group of atams or molecules which can be used to provide a detectable (preferably quantifiable) signal, and which can be attached to a nucleic acid or protein. Labels may provide signals detectable by fluorescence, radioactivity, colorimetry, gravimetry, X-ray diffraction or absorption, magnetism, enzymatic activity, and the like.

As used herein, two nucleic acid molecules are said to be capable of specifically hybridizing to one another if the two molecules are capable of forming an anti-parallel, double-stranded nucleic acid structure. A nucleic acid molecule is said to be the “complement” of another nucleic acid molecule if the molecules exhibit complete complementarity, i.e. every nucleotide of one of the molecules is complementary to a corresponding nucleotide of the other molecule. Two nucleic acid molecules are said to be “minimally complementary” if they can hybridize to one another with sufficient stability to permit them to remain annealed to one another under at least conventional “low-stringency” conditions. Similarly, two nucleic acid molecules are said to be “complementary” if they can hybridize to one another with sufficient stability to permit them to remain annealed to one another under conventional “high-stringency” conditions. Conventional stringency conditions are described by Sambrook et al., Molecular Cloning, A Laboratory Manual, 2nd Ed., Cold Spring Harbor Press, Cold Spring Harbor, N.Y. (1989) and by Haymes et al., Nucleic Acid Hybridization, A Practical Approach, IRL Press, Washington, DC (1985). Departures from complete complementarity are therefore permissible, as long as such departures do not completely preclude the capacity of the molecules to form a double-stranded structure. Thus, in order for a nucleic acid molecule to serve as a primer or probe it need only be sufficiently complementary in sequence to be able to form a stable double-stranded structure under the particular solvent and salt concentrations employed.

Appropriate stringency conditions which promote DNA hybridization, for example, 6.0 X sodium chloride/sodium citrate (SSC) at about 45° C., followed by a wash of 2.0×SSC at 50° C., are known to those skilled in the art or can be found in Current Protocols in Molecular Biology, John Wiley & Sons, N.Y. (1989), 6.3.1-6.3.6. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or either the temperature or the salt concentration may be held constant while the other variable is changed.

In a preferred embodiment, an oligonucleotide of the present invention will specifically hybridize to one or more of the common second nucleic acid molecules set forth in SEQ ID NO: 1 through SEQ ID NO: 6 and SEQ ID NO: 29 through SEQ ID NO: 35 or complements thereof under moderately stringent conditions, for example at about 2.0×SSC and about 65° C., more preferably under high stringency conditions such as 0.2×SSC and about 65° C.

As used herein, a nucleic acid molecule, be it a naturally occurring molecule or otherwise, may be “substantially purified”, if the molecule is separated from substantially all other molecules normally associated with it in its native state. More preferably a substantially purified molecule is the predominant species present in a preparation. A substantially purified molecule may be greater than 60% free, preferably 75% free, more preferably 90% free, and most preferably 95% free from the other molecules (exclusive of solvent) present in the natural mixture. The term “substantially purified” is not intended to encompass molecules present in their native state.

A subset of the oligonucleotides of the present invention to be used with conventional detection and quantitation methods includes nucleic acid molecules that hybridize to regulatory molecules selected from the group consisting of promoter and enhancer elements, 5′ untranslated leader sequences, 3′ untranslated leader sequences and intron sequences. Another subset of the oligonucleotides of the present invention hybridize to a selectable or screenable marker. Still another subset of the oligonucleotides of the present invention hybridize to signal sequences. Yet another subset of the oligonucleotides of the present invention hybridize to vector backbone sequences.

In one embodiment of the invention, oligonucleotides which hybridize to promoter sequences are provided. A “promoter” as used herein refers to a DNA fragment responsible for regulating transcription of DNA into RNA. Promoters comprise the DNA sequence, usually found upstream (5′) to a coding sequence, that regulates expression of the downstream coding sequence by controlling production of messenger RNA (mRNA) by providing the recognition site for RNA polymerase and/or other factors necessary for inititiating transcription at the correct site. Promoters are commonly part of nucleic acid expression cassettes. A number of promoters which are active in plant cells have been described in the literature. These include the nopaline synthase, (NOS) promoter (Ebert et al., Proc. Natl. Acad. Sci. (U.S.A.) 84:5745-5749 (1987)), the octopine synthase (OCS) promoter (which are carried on tumor-inducing plasmids of Agrobacterium tumefaciens), the caulimovirus promoters such as the cauliflower mosaic virus (CaMV) 19S promoter (Lawton et al., Plant Mol. Biol. 9:315-324 (1987)) and the CaMV 35S promoter (Odell et al., Nature 313:810-812 (1985)), the figwort mosaic virus 35S-promoter, the light-inducible promoter from the small subunit of ribulose-1,5-bis-phosphate carboxylase (ssRUBISCO), the Adh promoter (Walker et al., Proc. Natl. Acad. Sci. (U.S.A.) 84:6624-6628 (1987)), the sucrose synthase promoter (Yang et al., Proc. Natl. Acad Sci. (U.S.A.) 87:4144-4148 (1990)), the R gene complex promoter (Chandler et al., The Plant Cell 1:1175-1183 (1989)) and the chlorophyll a/b binding protein gene promoter, etc. These promoters have been used to create DNA constructs which have been expressed in plants; see, e.g., PCT publication WO 84/02913. Promoters also may be identified for use in the current invention by screening a plant cDNA library for genes which are selectively or preferably expressed in the target tissues or cells.

For the purpose of expression in source tissues of a plant, such as the leaf, seed, root or stem, one may choose from a number of promoters for genes with tissue- or cell-specific or -enhanced expression. Examples of such promoters reported in the literature include the chloroplast glutamine synthetase GS2 promoter from pea (Edwards et al., Proc. Natl. Acad. Sci. (U.S.A.) 87:3459-3463 (1990)), the chloroplast fructose-1,6-biphosphatase (FBPase) promoter from wheat (Lloyd et al., Mol. Gen. Genet. 225:209-216 (1991)), the nuclear photosynthetic ST-LS1 promoter from potato (Stockhaus et al, EMBO J. 8:2445-2451 (1989)), the serine/threonine kinase (PAL) promoter and the glucoamylase (CHS) promoter from Arabidopsis thaliana. Also reported to be active in photosynthetically active tissues are the ribulose-1,5-bisphosphate carboxylase (RbcS) promoter from eastern larch (Larix laricina), the promoter for the cab gene, cab6, from pine (Yamamoto et al., Plant Cell Physiol. 35:773-778 (1994)), the promoter for the Cab-1 gene from wheat (Fejes et al., Plant Mol. Biol. 15:921-932 (1990)), the promoter for the CAB-1 gene from spinach (Lubberstedt et al., Plant Physiol. 104:997-1006 (1994)), the promoter for the cab1R gene from rice (Luan et al., Plant Cell. 4:971-981 (1992)), the pyruvate, orthophosphate dikinase (PPDK) promoter from maize (Matsuoka et al., Proc. Natl. Acad. Sci. (U.S.A.) 90:9586-9590 (1993)), the promoter for the tobacco Lhcb1*2 gene (Cerdan et al., Plant Mol. Biol. 33:245-255 (1997)), the Arabidopsis thaliana SUC2 sucrose-H+ symporter promoter (Truernit et al., Planta. 196:564-570 (1995)) and the promoter for the thylakoid membrane proteins from spinach (psaD, psaF, psae, PC, FNR, atpC, atpD, cab, rbcS). Other promoters for the chlorophyll a/b-binding proteins may also be utilized in the present invention, such as the promoters for LhcB gene and PsbP gene from white mustard (Sinapis alba; Kretsch et al., Plant Mol. Biol. 28:219-229 (1995)).

A number of promoters for genes with tuber-specific or -enhanced expression for plants are known, including the class I patatin promoter (Bevan et al., EMBO J. 8:1899-1906 (1986); Jefferson et al., Plant Mol. Biol. 14:995-1006 (1990)), the promoter for the potato tuber ADPGPP genes, both the large and small subunits, the sucrose synthase promoter (Salanoubat and Belliard, Gene. 60:47-56 (1987), Salanoubat and Belliard, Gene. 84:181-185 (1989)), the promoter for the major tuber proteins including the 22 kd protein complexes and proteinase inhibitors (Hannapel, Plant Physiol. 101:703-704 (1993)), the promoter for the granule bound starch synthase gene (GBSS) (Visser et al., Plant Mol. Biol. 17:691-699 (1991)) and other class I and II patatins promoters (Koster-Topfer et al., Mol Gen Genet. 219:390-396 (1989); Mignery et al., Gene. 62:27-44 (1988)).

Other plant promoters can also be used to express a protein or fragment thereof of the present invention in specific tissues, such as seeds or fruits. The promoter for β-conglycinin (Chen et al., Dev. Genet. 10:112-122 (1989)) or other seed-specific promoters such as the napin and phaseolin promoters, can be used. The zeins are a group of storage proteins found in maize endosperm. Genomic clones for zein genes have been isolated (Pedersen et al., Cell 29:1015-1026 (1982)) and the promoters from these clones, including the 15 kD, 16 kD, 19 kD, 22 kD, 27 kD and alpha genes, could also be used. Other promoters known to function, for example, in maize include the promoters for the following genes: waxy, Brittle, Shrunken 2, Branching enzymes I and II, starch synthases, debranching enzymes, oleosins, glutelins and sucrose synthases. A particularly preferred promoter for maize endosperm expression is the promoter for the glutelin gene from rice, more particularly the Osgt-1 promoter (Zheng et al., Mol. Cell Biol. 13:5829-5842 (1993)). Examples of promoters suitable for expression in wheat include those promoters for the ADPglucose pyrosynthase (ADPGPP) subunits, the granule bound and other starch synthase, the branching and debranching enzymes, the embryogenesis-abundant proteins, the gliacdins and the glutenins. Examples of such promoters in rice include those promoters for the ADPGPP subunits, the granule bound and other starch synthase, the branching enzymes, the debranching enzymes, sucrose synthases and the glutelins. A particularly preferred promoter is the promoter for rice glutelin, Osgt-1. Examples of such promoters for barley include those for the ADPGPP subunits, the granule bound and other starch synthase, the branching enzymes, the debranching enzymes, sucrose synthases, the hordeins, the embryo globulins and the aleurone specific proteins.

Root specific promoters may also be used. An example of such a promoter is the promoter for the acid chitinase gene (Samac et al., Plant Mol. Biol. 25:587-596 (1994)). Expression in root tissue could also be accomplished by utilizing the root specific subdomains of the CaMV35S promoter that have been identified (Lam et al., Proc. Natl. Acad. Sci. (U.S.A.) 86:7890-7894 (1989)). Other root cell specific promoters include those reported by Conkling et al. (Conkling et al., Plant Physiol. 93:1203-1211 (1990)).

Additional promoters that may be utilized are described, for example, in U.S. Pat. Nos. 5,378,619; 5,391,725; 5,428,147; 5,447,858; 5,608,144; 5,608,144; 5,614,399; 5,633,441; 5,633,435; and 4,633,436. In addition, a tissue specific enhancer may be used (Fromm et al., The Plant Cell 1:977-984 (1989)).

Examples of suitable promoters for directing the transcription of a first transgenic nucleic acid molecule in a fungal host include promoters obtained from the genes encoding Aspergillus oryzae TAKA amylase, Rhizomucor miehei aspartic proteinase, Aspergillus niger neutral alpha-amylase, Aspergillus niger acid stable alpha-amylase, Aspergillus niger or Aspergillus awamori glucoamylase (glaA), Rhizomucor miehei lipase, Aspergillus oryzae alkaline protease, Aspergillus oryzae triose phosphate isomerase, Aspergillus nidulans acetamidase and hybrids thereof. In a yeast host, a useful promoter is the Saccharomyces cerevisiae enolase (eno-1) promoter. Particularly preferred promoters are the TAKA amylase, NA2-tpi (a hybrid of the promoters from the genes encoding Aspergillus niger neutral alpha -amylase and Aspergillus oryzae triose phosphate isomerase) and glaA promoters.

Suitable promoters for mammalian cells are also known in the art and include viral promoters such as that from Simian Virus 40 (SV40) (Fiers et al., Nature 273:113 (1978)), Rous sarcoma virus (RSV), adenovirus (ADV) and bovine papilloma virus (BPV).

Suitable promoters for insect cells are also known in the art and include baculovirus promoter (Smith and Summers, U.S. Pat. No., 4,745,051). derived from any of the over 500 baculoviruses generally infecting insects, such as for example the Orders Lepidoptera, Diptera, Orthoplera, Coleoptera and Hymenoptera, including for example but not limited to the viral DNAs of Autographa californica MNPV, Bombyx mori NPV, Trichoplusia ni MNPV, Rachiplusia ou MNPV or Galleria mellonella MNPV.

Examples of promoters suitable for use with bacterial hosts include the alpha-lactamase and lactose promoter systems (Chang et al., Nature 275:615 (1978); Goeddel et al., Nature 281:544; (1979)), the arabinose promoter system (Guzman et al., J. Bacteriol. 174:7716-7728 (1992)), alkaline phosphatase, a tryptophan (trp) promoter system (Goeddel, Nucleic Acids Res. 8:4057 (1980); EP 36,776) and hybrid promoters such as the tac promoter (deBoer et al., Proc. Natl. Acad. Sci. (USA) 80:21-25 (1983)). However, other known bacterial inducible promoters are suitable (Siebenlist et al., Cell 20:269 (1980)).

Promoters for use in bacterial systems also generally contain a Shine-Dalgarno (S.D.) sequence operably linked to the DNA encoding the polypeptide of interest. The promoter can be removed from the bacterial source DNA by restriction enzyme digestion and inserted into the vector containing the desired DNA.

Examples of suitable promoters for directing the transcription of a nucleic acid construct of the invention in an algal host include light harvesting protein promoters obtained from photosynthetic organisms, Chlorella virus methyltransferase promoters, CaMV 35 S promoter, PL promoter from bacteriophage λ, nopaline synthase promoter from the tDNA plasmid of Agrobacterium tumefaciens, and bacterial trp promotor.

In another embodiment of the invention, oligonucleotides which hybridize to 5′ non-translated leader sequence are used. 5′ non-translated leader sequences are characterized as that portion of the mRNA molecule which most typically extends from the beginning of the mRNA molecule (5′ CAP site which is a methylated guanosine nucleotide) to the AUG protein translation initiation codon. For most eukaryotic mRNAs, translation initiates with the binding of the CAP binding protein to the mRNA cap. This is then followed by the binding of several other translation factors, as well as the 43S ribosome pre-initiation complex. This complex travels down the mRNA molecule while scanning for an AUG initiation codon in an appropriate sequence context. Once this has been found and with the addition of the 60S ribosomal subunit, the complete 80S initiation complex inititates protein translation. A second class of mRNAs have been identified which possess distinct translation initiation features. Translation from these mRNAs initiates in a CAP-independent manner and is believed to initiate with the ribosome binding to internal portions of the 5′ non-translated leader sequence.

The efficiency of translation initiation can be influenced by features of the 5′ non-translated leader sequence, therefore identification and optimization of 5′ leader sequences can provide enhanced levels of gene expression in transgenic plants. For example, some studies have investigated the use of plant virus 5′ non-translated leader sequences for their effects on plant gene expression (Gallie et al., NAR 14:8693-8711, (1987); Jobling and Gehrke, Nature 325:622-625, (1987); Skuzeski et al., Plant mol. Bio. 15: 65-69, (1990). Increases in gene expression have been reported using the Tobacco Mosaic Virus (TMV) Omega leader. When compared with other viral leader sequences, such as the Alfalfa Mosaic Virus (AMV) RNA 4 leader, two to three fold improvements in the levels of gene expression were observed using the TMV Omega leader sequence (Gallie et al., 1987); (Skuzeski et al, 1990) Non-translated 5′ leader sequences associated with heat shock protein genes have also been demonstrated to significantly enhance gene expression in plants (see for example U.S. Pat. No. 5,362,865).

Most 5′ non-translated sequences of m-RNA are A-U rich and are predicted to lack significant secondary structure. One of the early steps in translation initiation is the relaxing or unwinding of the secondary mRNA structure (Sonenberg, Curr. Top. Micro. And Imm. 161:23-47, (1990). Messenger RNA leader sequences with negligible secondary mRNA structure may not require this additional unwinding step and may therefore be more accessible to the translation inititation components. Introducing sequences which can form stable secondary structures can reduce the level of gene expression (Kozak, Mol. And Cell Bio. 8:2737-2744 (1998); Pelletier and Aonenberg, Cell 40:515-526, (1985). The ability of a 5′ non-translated leader sequence to interact with translation components may play a key role in affecting the levels of subsequent gene expression.

The 5′ non-translated region may be associated with a gene from a source that is native or that is heterologous with repect to the other non-translated and/or translated elements present on the recombinant gene. Examples of 5′ non-translated sequences encoding heat shock proteins, fructose-1,6-bisphosphatases, chlorophyll a/b binding proteins, peroxidases, tubulins and amylases are reported in WO 00/11200.

In another embodiment of the invention, oligonucleotides hybridize to ribosomal binding domains. Insertion of ribosomal binding elements into, for example, vectors that contain promoters recognized by phage RNA polymerases in conjunction with the vaccinia virus-bacteriophage T7 expression system produce RNAs without cap structures at their 5′ end whose translation is greatly improved (Martinez-Salez, Current Opinion in Biotechnology: 10:458-464 (1999).

In another emobodiment of the invention, oligonucleotides hybridize to intervening sequences. Intervening sequences herein referred to as introns are also capable of increasing gene expression. Introns can improve the efficiency of mRNA processing. A number of introns have been reported to increase gene expression, particularly in monocots. In one report, the presence of the catalase intro I (Takanka, Nucl. Acid Res. 18:6767-6770 (1990) isolated from castor beans resulted in an increase in gene expression in rice but not in tobacco when using GUS as a marker gene. Still further improvements have been achieved, especially in monocot plants, by gene constructs which have introns in the 5′ non-translated leader positioned beween the promter and the structural coding sequence. For example, Callis et al., Genes and Develop. 1:1183-1200, (1987) reported that the presence of alcohol dehydrogenase (Adh-1) introns or Bronze-1 introns resulted in higher levels of expression. Mascarenkas et al., Plant mol. Biol. 15:913-920 (1990) reported a 12-fold enhancement of CAT expression by use of the Adh intron. Other introns suitble for use in DNA molecules include, but are not limited to, the sucrose synthase intron (Vasil et al., Bio/Technology 10:667 (1992), the TMV omega intron (Gallie et al., The Plant Cell 1:301-311 (1989), the maize hsp70 intron (U.S. Pat. No. 5,593,874 and U.S. Pat. No. 5,859,347), and the rice actin intron (McElroy et al., Plant Cell 2:163-171(1990).

In another embodiment of the invention, oligonucleotides hybridize to 3′ untranslated sequences. Untranslated sequences located at the 3′ end of a gene can also influence expression levels. A 3′ non-translated region comprises a region of the mRNA generally beginning with the translation termination codon and extending at least beyond the polyadenylation site. Ingelbrecht et al., Plant Cell 1:671-80, (1989) evaluated the importance of these elements and found large differences in expression in stable plants depending on the source of the 3′ non-translated region. Using 3′ non-translated regions associated with octopine synthase, 2S seed protein from Arabidopsis, samll subunit of rbsS from Arabidopsis extensin from carrot, and chalcone synthase from Antirrhinium, a 60 fold difference was observed between the best-expressing construct (which contained the rbsS 3′ non-translated region) and the lowest -expressing construct (which contained the caalcone synthase 3′ region). The 3′ non-translated region of the nopaline synthase gene of the T-DNA in Agrobacterium tumefaciens (3′ nos) (WO 00/11200) has also been used as a terminator region for expression of genes in plants.

In another emobodiment of the invention, oligonucleotides hybridize to marker sequences. Examples of such markers include, but are not limited to, a neo gene (Potrykus et al., Mol. Gen. Genet. 199:183-188 (1985)) which codes for kanamycin resistance and can be selected for using kanamycin; a bar gene which codes for bialaphos resistance; a mutant EPSP synthase gene (Hinchee et al., Bio/Technology 6:915-922 (1988)) which encodes glyphosate resistance; a nitrilase gene which confers resistance to bromoxynil (Stalker et al., J. Biol. Chem. 263:6310-6314 (1988)); a mutant acetolactate synthase gene (ALS) which confers imidazolinone or sulphonylurea resistance (European Patent Application 154,204 (Sep. 11, 1985)); and a methotrexate resistant DHFR gene (Thillet et al., J. Biol. Chem. 263:12500-12508 (1988)).

Screenable markers may also be used. Exemplary screenable markers include a β-glucuronidase or uidA gene (GUS) which encodes an enzyme for which various chromogenic substrates are known (Jefferson, Plant Mol. Biol, Rep. 5:387-405 (1987); Jefferson et al., EMBO J. 6:3901-3907 (1987)); an R-locus gene, which encodes a product that regulates the production of anthocyanin pigments (red color) in plant tissues (Dellaporta et al., Stadler Symposium 11:263-282 (1988)); a β-lactamase gene (Sutcliffe et al., Proc. Natl. Acad. Sci. (U.S.A.) 75:3737-3741 (1978)), a gene which encodes an enzyme for which various chromogenic substrates are known (e.g., PADAC, a chromogenic cephalosporin); a luciferase gene (Ow et al., Science 234:856-859 (1986)); a xylE gene (Zukowsky et al., Proc. Natl. Acad. Sci. (U.S.A.) 80:1101-1105 (1983)) which encodes a catechol dioxygenase that can convert chromogenic catechols; an α-amylase gene (Ikatu et al., Bio/Technol. 8:241-242 (1990)); a tyrosinase gene (Katz et al., J. Gen. Microbiol. 129:2703-2714 (1983)) which encodes an enzyme capable of oxidizing tyrosine to DOPA and dopaquinone which in turn condenses to melanin; an α-galactosidase, which will turn a chromogenic α-galactose substrate.

Included within the terms “selectable or screenable marker genes” are also genes which encode a secretable marker whose secretion can be detected as a means of identifying or selecting for transformed cells. Examples include markers which encode a secretable antigen that can be identified by antibody interaction, or even secretable enzymes which can be detected catalytically. Secretable proteins fall into a number of classes, including small, diffusible proteins which are detectable, (e.g., by ELISA), small active enzymes which are detectable in extracellular solution (e.g., α-amylase, β-lactamase, phosphinothricin transferase), or proteins which are inserted or trapped in the cell wall (such as proteins which include a leader sequence such as that found in the expression unit of extension or tobacco PR-S). Other possible selectable and/or screenable marker genes will be apparent to those of skill in the art.

In another embodiment of the invention, oligonucleotides hybridize to vector backbone sequences. Vectors for use in transgenic nucleic acid molecule transformation may include any vectors which can be conveniently subjected to recombinant DNA procedures or those which may bring about the expression of the nucleic acid sequence. The choice of vector will typically depend on the compatibility of the vector with the host cell into which the vector is to be introduced and the size of the nucleic acid molecule which is to be inserted. A vector system may be used. A vector system may contain a single vector or plasmid or two or more vectors or plasmids which together contain the total DNA to be introduced into the host.

Vector systems suitable for introducing transforming DNA into a host plant cell include but are not limited to Agrobacterium-mediated plant integrating vectors, binary artificial chromosome (BIBAC) vectors (Hamilton et al., Gene 200:107-116 (1997)); and transfection with RNA viral vectors (Della-Cioppa et al., Ann. N.Y. Acad. Sci. (1996), 792 (Engineering Plants for Commercial Products and Applications), 57-61). Additional vector systems also include plant selectable YAC vectors such as those described in Mullen et al., Molecular Breeding 4:449-457 (1988).

Examples of vectors suitable for transformation in other organisms include viral replicons such as the vaccinia virus (see, for example, Mackett et al, J. Virol. 49:857 (1984); Chakrabarti et al., Mol. Cell. Biol. 5:3403 (1985); Moss, In: Gene Transfer Vectors For Mammalian Cells (Miller and Calos, eds., Cold Spring Harbor Laboratory, N.Y., p. 10, (1987)), baculovirus expression vectors (BEVs) (Doerfler, Curr. Top. Microbiol. Immunol. 131:51-68 (1968); Luckow and Summers, Bio/Technology 6:47-55 (1988a); Miller, Annual Review of Microbiol. 42:177-199 (1988); Summers, Curr. Comm. Molecular Biology, Cold Spring Harbor Press, Cold Spring Harbor, N.Y. (1988)) and pBR322, is derived from an E. coli species (see, e.g., Bolivar et al., Gene 2:95 (1977)).

In another embodiment of the invention, oligonucleotides hybridize to a signal sequence. Signal sequences, when translated into proteins, enable the protein of the foreign gene to be sent to specific parts of the cell. Foreign genes encoding protein or fragments may be expressed along with the expression of a signal sequence or other polypeptide having a specific cleavage site at the N-terminus of the mature polypeptide. In general, the signal sequence may be a component of the vector, or it may be a part of the foreign gene that is inserted into the vector. The heterologous signal sequence selected should be one that is recognized and processed (i.e., cleaved by a signal peptidase) by the host cell, e.g. the alkaline phosphatase, penicillinase, lpp, or heat-stable enterotoxin II leaders.

First transgenic nucleic acid molecules and the vectors which contain them can be transformed into cells by a variety of means. Technology for introduction of DNA into cells is well known to those of skill in the art. General methods for delivering a gene into cells have been described: (1) chemical methods (Graham and van der Eb, Virology 54:536-539 (1973)); (2) physical methods such as microinjection (Capecchi, Cell 22:479-488 (1980)), electroporation (Wong and Neumann, Biochem. Biophys. Res. Commun. 107:584-587 (1982); Fromm et al., Proc. Natl. Acad. Sci. (U.S.A.) 82:5824-5828 (1985); U.S. Pat. No. 5,384,253); the gene gun (Johnston and Tang, Methods Cell Biol. 43:353-365 (1994)); (3)viral vectors (Clapp, Clin. Perinatol. 20:155-168 (1993); Lu et al., J. Exp. Med. 178:2089-2096 (1993); Eglitis and Anderson, Biotechniques 6:608-614 (1988)); and (4) receptor-mediated mechanisms (Curiel et al., Hum. Gen. Ther. 3:147-154 (1992), Wagner et al., Proc. Natl. Acad. Sci. (USA) 89:6099-6103 (1992)).

In another alternative embodiment, plastids (i.e. cellular organelles such as chloroplasts) can be stably transformed. Methods disclosed for plastid transformation in higher plants include the particle gun delivery of DNA containing a selectable marker and targeting of the DNA to the plastid genome through homologous recombination (Svab et al., Proc. Natl. Acad. Sci. (U.S.A.) 87:8526-8530 (1990); Svab and Maliga, Proc. Natl. Acad. Sci (U.S.A.) 90:913-917 (1993); Staub and Maliga, EMBO J. 12:601-606 (1993); U.S. Pat. Nos. 5,451,513 and 5,545,818).

A transformation method unique to some plants is called Agrobacterium-mediated transfer. The use of Agrobacterium-mediated plant integrating vectors to introduce DNA into plant cells is well known in the art. See, for example the methods described by Fraley et al., Bio/Technology 3:629-635 (1985) and Rogers et al., Methods Enzymol. 153:253-277 (1987). The region of DNA to be transferred into the host genome is defined by the tDNA border sequences in Agrobacterium-mediated plant integrating vectors and intervening DNA is usually inserted into the plant genome as described (Spielmann et al., Mol. Gen. Genet. 205:34 (1986)).

Modern Agrobacterium transformation vectors are capable of replication in E. coli as well as Agrobacterium, allowing for convenient manipulations as described (Klee et al., In: Plant DNA Infectious Agents, Hohn and Schell (eds.), Springer-Verlag, New York, pp. 179-203 (1985).

With reference to Table 1 preferred template sequences for such primers are fragments of common nucleic acid moleucles found in transgenic events such as the promoters, markers, tDNA border regions, 3′ and 5′ regions, having a sequence selected from any one of SEQ ID NO: 1 through SEQ ID NO: 6 and SEQ ID NO: 29 through SEQ ID NO: 35 or complements thereof. More particularly illustrative oligonucleotide primers include the nucleic acid molecules having a sequence of SEQ ID NO: 7 and 8 (i.e. forward and reverse primers for the 3′ untranslated region of the pea rbcS gene of SEQ ID NO: 2); SEQ ID NO: 10 and 11 (i.e. forward and reverse primers for the 3′ untranslated region of the NOS gene of SEQ ID NO: 35); SEQ ID NO: 13 and 14 (i.e. forward and reverse primers for the left tDNA border of SEQ ID NO: 4); SEQ ID NO: 16 and 17 (i.e. forward and reverse primers for the 3′ untranslated region of the NOS gene of SEQ ID NO: 35); SEQ ID NO: 19 and 20 (i.e. forward and reverse primers for the NPTII gene of SEQ ID NO: 3); SEQ ID NO: 23 and 24 (i.e. forward and reverse primers for the 3′ untranslated region of the NPTII gene of SEQ ID NO: 3); SEQ ID NO: 26 (i.e. forward primer for the petunia 5′ UTR leader sequence from the HSP70 gene of SEQ ID NO: 5); and SEQ ID NO: 28 (i.e. reverse primer for the 3′ untranslated region of the pea rbcS gene of SEQ ID NO: 2).

Also shown in Table 1 are the nucleic acid sequences for labeled oligonucleotide probes useful for detecting the presence or absence of surrogate nucleic acid molecules. Illustrative probes include those with the sequence of SEQ ID NO: 9 (i.e. for hybridizing to the 3′ untranslated region of the pea rbcS gene of SEQ ID NO: 2); SEQ ID NO: 12 (i.e. for hybridizing to the 3′ untranslated region of the NOS gene of SEQ ID NO: 35); SEQ ID NO: 15 (i.e. for hybridizing to the left tDNA border of SEQ ID NO: 4); SEQ ID NO: 18 (i.e. for hybridizing to the 3′ untranslated region of the NOS gene of SEQ ID NO: 35); SEQ ID NO: 21, 22 and 25 (i.e. for hybridizing to the NPTII gene of SEQ ID NO: 3); and SEQ ID NO: 27 (i.e. for hybridizing to the petunia 5′ UTR leader sequence from the HSP70 gene of SEQ ID NO: 5)

TABLE 1

| SEQ ID NO: | Description of sequence |
|---|---|
| 1. | 35S Cauliflower mosaic virus promoter |
| 2. | 3′ untranslated region of Pisum sativum rbcS gene |
| 3. | NPTII gene (kanamycin resistance) |
| 4. | left tDNA border |
| 5. | Petunia 5′UTR leader sequence from HSP70 gene |
| 6. | NOS promoter |
| 7. | forward primer for SEQ ID NO:2. |
| 8. | reverse primer (1) for SEQ ID NO:2. |
| 9. | probe for SEQ ID NO:2. |
| 10. | forward primer (1) for SEQ ID NO:35. |
| 11. | reverse primer (1) for SEQ ID NO:35. |
| 12. | probe (1) for SEQ ID NO:35. |
| 13. | forward primer for SEQ ID NO:4. |
| 14. | reverse primer for SEQ ID NO:4. |
| 15. | probe for SEQ ID NO:4. |
| 16. | forward primer (2) for SEQ ID NO:6. |
| 17. | reverse primer (2) for SEQ ID NO:6. |
| 18. | probe (2) for SEQ ID NO:6. |
| 19. | forward primer (1) for SEQ ID NO:3. |
| 20. | reverse primer (1) for SEQ ID NO:3. |
| 21. | probe (1a) for SEQ ID NO:3. |
| 22. | probe (1b) for SEQ ID NO:3. |
| 23. | forward primer (2) for SEQ ID NO:3. |
| 24. | reverse primer (2) for SEQ ID NO:3. |
| 25. | probe (2) for SEQ ID NO:3. |
| 26. | forward primer for SEQ ID NO:5. |
| 27. | probe for SEQ ID NO:5. |
| 28. | reverse primer (2) for SEQ ID NO:2 |
| 29. | chloramphenical-resistance gene |
| 30. | ampicillan resistance gene |
| 31. | Adh promoter |
| 32. | wheat fructose 1,6-biphosphatase 5′ untranslated leader |
| 33. | 3′ untranslated sequence from the wheat ubiquitin gene |
| 34. | right tDNA border |
| 35. | 3′ untranslated region from nopaline synthase gene |

b) Transgene Detection and Quantitation Methodologies

The oligonucleotides of this invention, described above, may be used with conventional detection and quantitation methods. These methods are either based on hybridization between the oligonucleotides of this invention followed by amplification of all or part of the second transgenic nucleic acid molecule or on hybridization of the oligonucleotides of this invention without amplification of second transgenic nucleic acid molecules.

In one embodiment of the invention, a first transgenic nucleic acid molecule is detected or quantitated by 1) amplifying a second transgenic nucleic acid molecule and then 2) detecting the amplification product. DNA can be extracted from a sample, if desired, using any of the well known methods familiar to those of skill in the art (Current Protocols in Molecular Biology Ausubel, et al., eds., John Wiley & Sons, N.Y. (1989), and supplements through September (1998). Amplification may be carried out by any method known to those of skill in the art. The preferred method is the polymerase chain reaction (PCR), the details of which are provided in U.S. Pat. No. 4,683,195, U.S. Pat. No. 4,965,188, all to Mullis et al.

Briefly, the PCR exploits certain features of DNA replication. An enzyme, DNA polymerase, uses single-stranded DNA as a template for the synthesis of a complementary new strand. These single-stranded DNA templates can be produced by heating double-stranded DNA to temperatures near boiling. DNA polymerase also requires a small section of double-stranded DNA to initiate (“prime”) synthesis. Therefore, the starting point for the DNA synthesis can be specified by supplying a primer that anneals to the template at that point.

Both DNA strands can serve as templates for synthesis provided a primer is provided for each strand. For a PCR, the primers are chosen to flank the region of DNA that is to be amplified so that the newly synthesized strands of DNA, starting at each primer, extend beyond the position of the primer on the opposite strand. Therefore, new primer binding sites are generated on each newly synthesized DNA strand. The reaction mixture is again heated to separate the original and newly synthesized strands which are then available for further cycles of primer hybridization, DNA synthesis and strand separation. The net result of a PCR is that by the end of n cycles, the reaction contains a theoretical maximum of 2ⁿ double-stranded DNA molecules that are copies of the DNA sequence between the primers.

PCR often reaches a plateau phase, however, where the amount of amplified product is not reflective of the amount of template present in the initial reaction. This plateau phase may be caused by many factors including shortage of primer or nucleotide substrates. Methods using the PCR have been described which overcome this deficency when quantitation of the initial template is desired (e.g. PCR Primer: A Laboratory Manual Dieffenbach, C and D. Gabriela, eds, Cold Spring Harbor Laboratory Press, Cold Spring Harbor, N.Y. (1995)) and some of these are described below.

For example transgene quantitation can be determined by quantitative PCR. There are may variations of quantitative PCR (e.g. Ferre F., PCR Methods and Appl. 2:1-9 (1992) ). One illustrative example is given here. Two primers are designed for a nucleic acid sequence of interest. The primers are end labeled with ³²P. Aliquots of the reaction are removed during the PCR. A range of cycle points between 16 and 26 cycles is usually used. Samples from each PCR reaction cycle point are loaded into a nondenaturing gel. After the gel is run and stained with an intercalating dye, the bands are isolated from the gel and placed in an Eppendorf tube for counting. Counts are determined by Cerenkov counting and log counts plotted against cycle point. The slope of this plot determines the efficiency of the enzyme in the reaction. The amount of DNA before PCR amplificiation (log DNA₀) can then be calculated from the equation: log DNAₙ=log DNA₀+n log (1+R) where log DNAₙ is the amount of incorporated primer at cycle number n; log DNA₀ is the amount of incorporated primer at the first cycle; n is the cycle number, R is the efficiency of Taq polymerase; and log (1+R) is the slope of the plot.

Copy number of a nucleic acid sequence of interest may also be determined using ,competitive quantitative PCR. There are many variations of competitive quantitative PCR (e.g. Wang et al., Proc. Natl. Acad. Sci. USA 86: 9717-9721, (1989)) An illustrative example is here described. An artificially introduced DNA molecule is added, either to the extraction step or the PCR step, in a known concentration (i.e an exogenous control). (Chelly et al., Nature 333:858-860 (1988)). The exogenous control is amplified with the same primers as the target sequence to more accurately reflect target sequence amplification efficiency relative to the exogenous control. (WO/93/02215; WO 92/11273; U.S. Pat. No. 5,213,961 and U.S. Pat. No. 5,219,727). The detection of amplified products following competitive quantitative PCR must provide a method of distinguishing the added control standard from the target nucleic acid sequence. Exogenous controls can be designed so as to be distinguishable by size of the amplified product as visualized on an agarose gel (Scadden et al., J. Infect Dis 165:1119-1123, (1992); Piatiak et al, Biotechniques 14:70-80 (1993)) or by introducing an internal restriction site through mutagenesis, wherein the restriction fragments are again detected on an agarose gel (Becker-Andre and Hahlbrock Nucleic Acid Res. 17:9437-9446 (1989)); Steiger et al J. Virol Methods 34: 149-160 (1991)). Additional detection methods may be used (Mulder et al J. Clin Microbiol. 32:292-300, (1994))

Other technologies for the amplification of nucleic acids have been described, most of which are based upon isothermal amplification strategies as opposed to the temperature cycling required for PCR. These strategies include, for example, Strand Displacement Amplification (SDA) (U.S. Pat. Nos. 5,455,1666 and 5,457,027 and Nucleic Acid Sequence-Based Amplificiation (NASBA) (U.S. Pat. No. 5,130,238; European Patent 525882 to Kievits. Each of these amplification technologies are similar in that they employ the use of short, deoxyribonucleic acid primers to define the region of amplification, regardless of the enzymes or specific conditions used.

Amplification is carried out using a DNA polymerase. As defined herein “DNA polymerase” refers to a family of enzymes known to those skilled in the art. DNA polymerases are enzymes that recognize the junction between single-stranded and double-stranded nucleic acids created by the hybridization of primer to a second nucleic acid molecule. DNA polymerases useful in the present invention include, but are not limited to, Taq polymerase, T4 DNA polymerase, T7 DNA polymerase, Thiredoxin, thermostable DNA polymerase from Pyrococcus woesei, and Klenow Fragment DNA polymerase. Preferred DNA polymerases have 5′-3′ exonuclease activity. 5′-3′ exonuclease activity” refers to the removal of nucleotide sequences in the 5′-3′ direction by a polymerase as synthesis occurs.

In a preferred embodiment of the invention, amplification is carried out using a 5′ nuclease assay. As used herein, a “5′ nuclease assay” is carried out with a polymerase having 5′-3′ exonuclease activity. Additionally, a forward primer, a reverse primer and a corresponding probe are used. The probe is labeled with a flurophore reporter dye at the 5′ end and a fluorophore quencher dye is at the 3′ end. All three oligonucleotides are included in the amplification process. The forward and reverse primers anneal to a second transgenic nucleic acid molecule and the probe anneals between the forward and reverse primers. As extension of the forward primer occurs, the reporter dye is cleaved by the action of the polymerase. The separation of the reporter dye and the quencher dye results in an increase in signal which indicates the presence of a second transgenic nucleic acid molecule. The primers and probe anneal at each PCR cycle and cleavage of the reporter dye occurs at each PCR cycle. This method is quantitative since the release of the flurogenic tag from the 5′ end of the probe is proportional to the copy number of the second transgenic nucleic acid molecule. See U.S. Pat. Nos. 5,210,015; 5,538,848; 5,723,591; 5,876,930; 5,925,517; 5,945,283; 5,962,233; and 6,030,787, all of which are incorporated herein by reference in their entireties.

In another embodiment of the invention, a second transgenic nucleic acid molecule is a mRNA molecule. RNA may be extracted from a sample by any of the methods well known to those of skill in the art. In this embodiment, a reverse transcriptase reaction step preceeds the PCR step (i.e. RT-PCR), after which the amplified product is detected. A single-strand complementary DNA, (cDNA) of the mRNA is produced through the action of a retroviral enzyme, reverse transcriptase, e.g. AMV reverse transcriptase, MMLV reverse transcriptase, “Tth” DNA polymerase, and the like. A primer is required to initiate cDNA synthesis. The primer anneals to the mRNA, and the cDNA is extended toward the 5′ end of the mRNA through the RNA-dependent DNA polymerase activity of reverse transcriptase. Random hexamer primers may be used which bind to all RNAs present in a sample. Similarly, primers may be used which consist solely of deoxythymidine residues (oligo(dT) and anneal to the polyadenylated 3′ tail found on most mRNAs.

Alternatively, a gene-specific primer can be used for the RT reaction. For some genes, especially rare messages, the use of sequence-specific primers increases specificity and decreases background associated with other types of primers. These primers can then be used for the subsequent PCR in conjunction with the corresponding gene-specific forward primer.

Following the RT reaction, the cDNA is amplified by PCR. PCR is usually carried out using an aliquot of the RT reaction or by adding the necessary PCR components directly to the RT reaction.

There are many methods and variations of them used for the quantitation of mRNA molecules using RT-PCR (Freeman et. al., BioTechniques 26:112-125 (1999)). These methods often require a standard. A wide range of DNA and RNA standards have been reported (Freeman et al., 1999). One commonly used standard is referred to as a homologous synthetic RNA standard. This type of standard can be defined as an in vitro-transcribed synthetic RNA that shares the same primer binding sites as the target RNA and has the same intervening sequence except for a small insertion, deletion or mutation to facilitate differentiation from the native signal during quantification. Homologous RNA standards are most likely to have the same or very similar PCR efficiencies as the target and an RNA standard is often better than a DNA standard because an RNA standard can control for variability during the RT step. Homologous RNA standards are generally created from the entire target sequence, or a portion of it and cloned into a plasmid containing an RNA polymerase promoter suitable for in vitro transcription. A small deletion or insertion or a mutation is designed in the standard so that the target and standard amplification products can be differentiated by size on an electrophoresis gel.

Two approaches exist for using co-amplified standards (Freeman et. al, 1999); competitive and non-competitive. In non-competitive RT-PCR, increasing series of standard amounts are co-amplified with equal amounts of total experimental RNA. This occurs under conditions in which there is no competition for the components in the PCR. The quantification is estimated on a linear-scaled graph. The amount of standard signal is plotted aginst the experimental signal. When the lines intersect, they reach the equivalence point, and quantification is achieved.

In competitive RT-PCR the standard competes with the target of interest for primers and enzyme, thus reducing the amount of the target of interest that is formed when the standard is in excess. As the amount of standard increases, the amount of the nucleic acid molecule of interest that is formed decreases. Quantification could be achieved from a graph of the log of standard signal/target signal vs. the log of input RNA standard, the amount of initial, nucleic acid molecule of interest can be determined at the equivalence point (Freeman et. al, 1999).

Another type of standard is an endogenous control standard. Endogenous controls are generally housekeeping genes (e.g. human glyceraldehyde-3-phosphate dehydrogenase (GADPH) cDNA). Housekeeping genes are ubiquitously expressed, have high expression levels, and their expression is constant at different times. Reporting expression levels relative to housekeeping genes whose expression does not change makes it possible to accurately asses gene expression levels across different experimental samples. Amplified products resulting from PCR, RT-PCR or any variation of these described above may be detected and quantitated by any of detection and quantitation techniques including traditional “end-point” measurements of product and “real-time” monitoring of product formation. Endpoint determinations analyze the reaction after it is completed, and real-time determinations monitor the reaction in a thermal cycler as it progresses. End-point product measurement include the use of fluorescent intercalating dyes. (e.g ethidium bromide or SYBER Green) of the amplified product or through measurement of incorporated radioactivity by autoradiography (see Freeman et. al, 1999 for other methods). Hybridization based protocols, such as Southern blots or fluorescence detection are also used. A third type of end-point product measurement uses solid-state approaches in which a bound enzyme produces fluorescence or luminescence (see Freeman et. al, 1999 for additional methods and details).

In the simplest embodiment of this invention, amplified products are detected by running them on an agarose gel which is then stained with an intercalating dye.

Real-time detection eliminates the need for post-PCR processing since detection occurs during each PCR cycle. Higuchi et al., Bio/Technology 10:413-417 (1992) and Ishiguro et al., Anal. Biochem 229:207-213 (1995) describe the use of various intercalaters to detect PCR amplification products. Higuchi et al., Bio/Technology 11:1026-1030 (1993) introduced the idea of real-time PCR product detection by measuring the increase in ethidium bromide intensity during amplification with a charge-coupled device (CCD) camera. Ishiguro et al. (1995) have also reported ‘real time’ PCR detection of hepatitis C virus RNA, using the intercalator YO-PRO-1. A ‘PCR monitor’, which partially consists of a modified laser excitation fluorescence detector and a thermal cycler, is used to detect the emission of a fluorescent intercalator during amplification.

Wittwer et al., BioTechniques 22:130-138 (1997) have illustrated the utilization of a 5′ nuclease assay for continuous fluorescence monitoring in capillary tubes. Samples are run in a ‘fluorescence temperature (hot air) cycler’ and the increase in fluorescence is monitored during the extension phase for each cycle. An amplification plot comparing cycle numbers and fluorescence ratio is generated to quantitate the amount of starting nucleic acid molecules.

Recently, Heid et al., Genome Res. 6: 986-984 (1996) Gibson et al. Genome Res. 6: 995-1001(1996) and Livak et al. PCR Methods and Applications 4:357-362 (1995) have described a real time detection method using the ABI 7700 system. The ABI PRISM™ 7700 Sequence Detector is comprised of a 96-well thermocycler, argon laser and CCD camera. During PCR, a dual-labeled oligo probe that is annealed to a target sequence is cleaved by the 5′-3′ exonuclease activity of the extending Taq polymerase, releasing a reporter dye located on the 5′ end of the probe (6-carboxy-fluorescein [FAM]) from a quencher dye located on the 3′ end of the probe (6-carboxy-tetramethyl-rhodamine [TAMRA]). An argon laser is used to excite electrons from the fluorescein reporter molecules. Emissions between 500 and 600 nm are captured through fiber optic cables and focused by a dicroic mirror into a spectrograph.

Light is separated based on wavelength across a CCD camera and the data analyzed by the software's algorithms. Emission intensities of the reactions are measured sequentially every seven seconds (for 25 milliseconds) and the intensities of reporter dye versus quencher dye emissions evaluated. Since the emission intensity of the quencher dye varies only minimally during the PCR, it is used to normalize variations in reporter dye emission intensities. A value termed Rn is calculated by the instrument software using the equation Rn=(Rn+)−(Rn−). (Rn+) is the emission intensity of the reporter divided by the emission intensity of the quencher during a specific amplification cycle, and (Rn−) is the emission intensity of the reporter divided by the emission intensity of the quencher prior to amplification. Therefore, Rn represents the amount of annealed probe cleaved by the 5′-3′ exonuclease activity of Taq polymerase during amplification. An average Rn for each cycle is calculated during the syntheis phase and is plotted versus cycle number, generating an amplification plot. The cycle number at which the Rn rises above baseline (termed Ct) is inversely proportional to the copy number of the original target template.

In a preferred embodiment of the invention the expression of a first transgenic nucleic acid molecule is detected and/or quantitated by hybridizing at least one oligonucleotide to a 3′ untranslated region. In a more preferred embodiment of the invention a primer pair and corresponding probe are designed which hybridize to a 3′ untranslated region and expression of a first transgenic nucleic acid molecule is detected and/or quantitated in a 5′ nuclease assay. In an even more preferred embodiment of the invention a primer pair and corresponding probe are designed which hybridize to a 3′ end of the Pisum sativum rbcS E9 gene and expression of a first transgenic nucleic acid molecule is detected and/or quantitated in a 5′ nuclease assay

There are additional detection and quantitation techniques well known in the art which do not require amplification. These techniques may be used in conjunction with this invention and include, but are not limited to, blotting methods such as Southern Blotting (DNA) or Northern Blotting (RNA) and RNAse protection assays the details of which can be found in Current Protocols in Molecular Biology Ausubel, et al., eds., John Wiley & Sons, N.Y. (1989), and supplements through September (1998).

This invention may be used in a variety of applications including but not limited to transformant selection, the detection of genetically modified products, microbial bioprocessing applications, and human gene therapy. For more details on these applications see Recombinant DNA Watson et. al., W. H. Freeman and Company (1992), the entirety of which is herein incorporated by reference.

It is to be understood that both the foregoing general description and detailed description are exemplary and explanatory only and are not restrictive of the invention claimed.

### EXAMPLE 1

This example illustrates how to detect and quantitate expression of a first transgenic nucleic acid molecule by hybridizing oligonucleotides to a second transgenic nucleic acid molecule.

Three hole punches of leaves from Arabidopsis thaliana are flash frozen in liquid nitrogen. The frozen tissue is subsequently freeze dried for a period of 48 hours. The freeze dried tissue is placed in a 1.4 ml tube with a glass bead (3 mm), capped; and pulverized into a fine powder using a Retsch model MM300 laboratory vibration mill. RNA is extracted according to the Qiagen™ (Valencia, Calif.) Rneasy Plant Mini kits (Catalogue number 74904). RT-PCR reactions and thermocycling conditions are according to the Taqman™ One Step RT-PCR Master Mix Reagents Kit (Perkin Elmer Applied Biosystems, Foster City, Calif.). Approximately 40 ng of total RNA is used per reaction with a final concentration of 300 nM of primer pair targeting SEQ ID NO: 3, the 3′ untranslated region of the Pisum sativum rbcS E9 gene. This 3′ untranslated region is used as the second nucleic acid molecule to detect the expression of a first transgenic nucleic acid molecule which may be any gene operably linked and co-expressed with it. The primers targeting this 3′ untranslated region are listed in SEQ ID NO: 7, SEQ ID NO: 8 and SEQ ID NO: 28. SEQ ID NO: 7 may be used with either SEQ ID NO: 8 or SEQ ID NO: 28. A final concentration of 200 nM of probe (SEQ ID NO: 9) is used along with a final concentration of 20 nM of 18S rRNA endogenous control primer and a final concentration of 50 nM endogenous 18S rRNA control probe. The probes are labeled at the 5′ end with FAM and on the 3′ end with TAMRA. Primers and probes are selected with Primer Express software Version 1.0 (PE Applied Biosystems) using default values.

Real time detection of RT-PCR is carried out using the ABI®7700 Sequence detection system from PE Applied Biosytems following the protocols found on http://www.pebio.com. The amount of the 3′untranslated region is determined by relative quantitation. The 18S rRNA endogenous control is used to normalize the expression of the 3′ untranslated region. The availability of distinguishable reporter dyes for the ABI®7700 Sequence detection system makes it possible to amplify and detect the target amplicon and the endogenous control amplicon in the same tube (i.e.multiplex PCR). A calibrator transgenic line is chosen preferably to compare individual experimental ΔCt values to generate ΔΔCt values. A calibrator transgenic line is one whose expression has been relatively quantitated using a different method such as Northern Blotting. The relative gene expression between the calibrator line and the experimental line containing the untranslated 3′ end of the Pisum sativum rbcS E9 gene is calculated as 2^{-ΔΔCt}.

### EXAMPLE 2

This example illustrates how to detect and quantitate transgene copy number of a first transgenic nucleic acid molecule by hybridizing oligonucleotides to a second transgenic nucleic acid molecule.

Three hole punches of leaves from Arabidopsis thaliana are flash frozen in liquid nitrogen. The frozen tissue is subsequently freeze dried for a period of 48 hours. The freeze dried tissue is placed in a 1.4 ml tube with a glass bead (3 mm), capped ;and pulverized into a fine powder using a Retsch model MM300 laboratory vibration mill. Genomic DNA is extracted according to the Qiagen™ Dneasy Plant Mini kit (Catalogue number 69104). Multiplex PCR reactions and thermocycling conditions are according to Taqman™ Universal PCR Master Mix Reagent kit (PE Applied Biosystems). Primer sets and probe sets are designed for the t-DNA left border region (SEQ ID NO: 13 to SEQ ID NO: 14 for the primers and SEQ ID NO: 15 for the probe). The probe is labeled at the 5′ end with FAM and at the 3′ end with TAMRA. Primers and probe are selected using Primer Express Version 1.0 (PE Applied Biosystems) default parameters.

Real time detection of PCR is carried out using the ABI®7700 Sequence detection system from PE Applied Biosytems following the protocols found on http://www.pebio.com. Copy number determination is achieved by relative quantitation. A ΔCt for an unknown is first normalized to an endogenous control. The endogenous control is specific for a gene of known copy number. Copy number is then estimated by subtracting the ΔCt of calibrator line(s) (i.e. a transgenic line whose transgene copy number has been previously determined by another method such as Southern blotting) from an unknown sample's ΔCt to generate ΔΔCt values. The transgene copy number in varous lines can be estimated by 2^{-ΔCt}.

### EXAMPLE 3

This example illustrates how to detect and quantitate transgene zygosity of a first transgenic nucleic acid molecule by hybridizing oligonucleotides to a second transgenic nucleic acid molecule. This method is generally applicable to any transgenic plant or line or population however it is preferred to determine zygosity on a plant, line or population previously shown to have a single copy of the transgene by using the methods described in Example 2.

Three hole punches of leaves from a transgenic Arabidopsis thaliana are flash frozen in liquid nitrogen. The frozen tissue is subsequently freeze dried for a period of 48 hours. The freeze dried tissue is placed in a 1.4 ml tube with a glass bead (3 mm), capped ;and pulverized into a fine powder using a Retsch model MM300 laboratory vibration mill. Genomic DNA is extracted according to the Qiagen™ Dneasy Plant Mini kit (Catalogue number 69104). Multiplex PCR reactions and thermocycling conditions are according to Taqman™ Universal PCR Master Mix Reagent kit (PE Applied Biosystems). Primer sets and probe sets are designed for the t-DNA left border region (SEQ ID NO: 13 to SEQ ID NO: 14 for the primers and SEQ ID NO: 15 for the probe). The probe is labeled at the 5′ end with FAM and at the 3′ end with TAMRA. Primers and probe are selected using Primer Express Version 1.0 (PE Applied Biosystems) default parameters.

Real time detection of PCR is carried out using the ABI®7700 Sequence detection system from PE Applied Biosytems following the protocols found on http://www.pebio.com. Zygosity determination is achieved by relative quantitation. A ΔCt for an unknown is first normalized to an endogenous control. The endogenous control is specific for a gene of known copy number and zygosity. Zygosity is then estimated by subtracting the ΔCt of calibrator line(s) (i.e. a transgenic line whose transgene zygosity has been previously determined by another method such as Southern blotting or segregation analysis) from an unknown sample's ΔCt to generate ΔΔCt values. The transgene zygosity in various lines can be estimated by 2^{-ΔΔCt}. Alternatively, the zygosity can be inferred without the use of a calibrator line by statistical analysis of the ΔCt values and separation into null, heterozygous and homozygous classes.

1-34. (canceled)

35. A method to detect expression of a first transgenic nucleic acid molecule in a sample having either (a) a detectable amount of mRNA transcribed from a second transgenic nucleic acid molecule or (b) a substantially non-detectable amount of said mRNA, said method comprising providing a complementary DNA of the mRNA, amplifying said complementary DNA and hybridizing said complementary DNA with at least one oligonucleotide designed to hybridize to said second transgenic nucleic acid molecule whereby said hybridizing indicates the expression of said first transgenic nucleic acid molecule in a sample.

36. A method according to claim 35 further comprising quantitation of mRNA transcribed from said second transgenic nucleic acid molecule.

37. A method according to claim 35 wherein said second transgenic nucleic acid molecule which is selected from the group consisting of signal sequences, 3′ UTR sequences and 5′ UTR sequences.

38. A method according to claim 35 wherein said second transgenic nucleic acid molecule is a 3′ untranslated sequence from the 3′ end of the Pisum sativum rbcS E9 gene.

39. A method according to claim 35 wherein said second transgenic nucleic acid molecule has a sequence of SEQ ID NO: 2.

40. A method according to claim 35 wherein the at least one oligonucleotide is a sequence which is a molecule selected from the group consisting of SEQ ID NO: 7 SEQ ID NO: 8, SEQ ID NO: 9 and SEQ ID NO: 28.

41. A method according to claim 35 wherein the amplifying is carried out by a method selected from the group consisting of PCR or RT-PCR.

42. A method according to claim 36 wherein the quantitation of mRNA is determined by a method selected from the group consisting of quantitative RT-PCR or competitive quantitative RT-PCR.

43. A method according to claim 35 wherein said second transgenic nucleic acid molecule comprises at least 100 base pairs of consecutive sequence having a sequence of SEQ ID NO: 2.

44. A method according to claim 35 wherein at least one oligonucleotide comprises at least 15 bases from or complementary to a consecutive sequence of SEQ ID NO: 2.

45. A method according to claim 35 wherein at least one oligonucleotide has a detectable label.

46. A method according to claim 45 wherein said label is selected from the group consisting of a fluorescent label, a digoxigenen-dUTP label, a biotin label, and a radiolabel.

47. A method according to claim 35 wherein said at least one oligonucleotide comprises a pair of oligonucleotide primers and an oligonucleotide probe designed to hybridize to said second transgenic nucleic acid molecule in a 5′ nuclease assay.

48. A method according to claim 47 wherein each of said primer pair used in said amplification comprises 15 to 30 bases identical or complementary to a consecutive sequence of a second transgenic nucleic acid molecule having a sequence selected from the group consisting of signal sequences, 3′ UTR sequences and 5′ UTR sequences and wherein said probe comprises 15 to 30 bases complementary or identical to a second transgenic nucleic acid molecule having a sequence selected from the group consisting of signal sequences, 3′ UTR sequences and 5′ UTR sequences.

49. A method according to claim 35 further comprising Southern Blotting, Northern Blotting or RNAse protection assay.

50. An amplification kit for the detection of a transgenic nucleic acid molecule comprising at least one primer pair and a corresponding labeled probe which hybridizes under stringent hybridization conditions to a nucleic acid molecule of a 3′ untranslated sequence of a 3′ end of the Pisum sativum rbcS E9 gene.

51. A method to detect expression of a first transgenic nucleic acid molecule in a sample having either (a) a detectable amount of mRNA transcribed from a second transgenic nucleic acid molecule or (b) a substantially non-detectable amount of said mRNA, said method comprising providing a complementary DNA of the mRNA, amplifying said complementary DNA and hybridizing said complementary DNA with at least one oligonucleotide designed to hybridize to said second transgenic nucleic acid molecule whereby said hybridizing indicates the expression of said first transgenic nucleic acid molecule in a sample and wherein said at least one oligonucleotide is a sequence which is a molecule selected from the group consisting of SEQ ID NO: 7 SEQ ID NO: 8, SEQ ID NO: 9 and SEQ ID NO: 28.

52. A method to detect expression of a first transgenic nucleic acid molecule in a sample having either (a) a detectable amount of mRNA transcribed from a second transgenic nucleic acid molecule or (b) a substantially non-detectable amount of said mRNA, said method comprising providing a complementary DNA of the mRNA, amplifying said complementary DNA and hybridizing said complementary DNA with at least one oligonucleotide designed to hybridize to said second transgenic nucleic acid molecule whereby said hybridizing indicates the expression of said first transgenic nucleic acid molecule in a sample and wherein said second transgenic nucleic acid molecule is the sequence of SEQ ID NO: 2.
